(19) United States
(12) Patent Application Publication
(10) Pub. No.: US 2025/0242142 A1
(43) Pub. Date: Jul. 31, 2025

(54) Tattoo Ink Mixing Device

(71) Applicants: Josiah Moyer, Wernersville, PA (US); Faith Moyer, Wernersville, PA (US)

(72) Inventors: Josiah Moyer, Wernersville, PA (US); Faith Moyer, Wernersville, PA (US)

(21) Appl. No.: 18/424,141

(22) Filed: Jan. 26, 2024

(51) Int. Cl. A61M 37/00; B01F 33/501; B01F 101/35

(52) U.S. Cl. CPC A61M 37/0084; B01F 33/50112; B01F 2101/35

(57) ABSTRACT

A tattoo ink mixing device for mixing and generating customized colors of tattoo inks includes a housing having an interior space. Each of a set of ink cartridges positionable in the interior space holds a respective one of a plurality of colored inks. Each of a plurality of cartridge pumps positioned in the interior space is fluidly couplable to a respective one of the set of ink cartridges. The plurality of cartridge pumps release a quantity of the respective one of the plurality of colored inks from the respective one of the set of ink cartridges. A mixing bowl positioned in the interior space is fluidly coupled with each of the plurality of cartridge pumps to receive the quantity of the respective one of the plurality of colored inks, which mixes with an additional quantity of an additional one of the plurality of colored inks thereby forming a mixed ink.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to ink blending machines and more particularly pertains to a new ink blending machine for mixing and generating customized colors of tattoo inks.

### (2) Description of Related Art Including Information Disclosed Under 37 CFR 1.97 and 1.98

The prior art relates to ink blending machines. The prior art discloses machines that can blend colors and pigments of inks, for example in printers or for color-matching paints. However, the prior art lacks disclosure of a machine for blending and producing customized shades of tattoo ink. The tattoo industry is a highly creative field, with artists constantly creating new designs. Tattoo artists can purchase inks in various colors, and often need to mix those colors together by hand to produce the particular shade that a client desires for their tattoo. Hand mixing can be a time-consuming task, and purchasing inks in every shade can be extremely expensive. Thus, there is a need for a machine that can blend together a standard array of colored inks to create a desired shade or pigment of tattoo ink.

### BRIEF SUMMARY OF THE INVENTION

An embodiment of the disclosure meets the needs presented above by generally comprising a housing having an interior space. A power source is positioned in the interior space. A processor is positioned in the interior space and is electrically coupled to the power source. A plurality of cartridges is positionable in the interior space. The plurality of cartridges includes a set of ink cartridges. Each of the set of ink cartridges holds a respective one of a plurality of colored inks. A plurality of cartridge pumps is positioned in the interior space and is electrically coupled to the processor. Each of the plurality of cartridge pumps is fluidly couplable to a respective one of the plurality of cartridges. The processor activates at least one of the plurality of cartridge pumps to release a quantity of the respective one of the plurality of colored inks from the respective one of the plurality of cartridges. A mixing bowl is positioned in the interior space. The mixing bowl is fluidly coupled with each of the plurality of cartridge pumps to receive the quantity of the respective one of the plurality of colored inks. The quantity of the respective one of the plurality of colored inks mixes with an additional quantity of an additional one of the plurality of colored inks in the mixing bowl thereby forming a mixed ink. A cup is removably positionable in the interior space. The cup is in fluidic communication with the mixing bowl when the cup is positioned in the interior space whereby the cup receives the mixed ink.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a tattoo ink mixing device according to an embodiment of the disclosure.

FIG. 2 is an exploded view of an embodiment of the disclosure.

FIG. 3 is a detailed view of an embodiment of the disclosure.

FIG. 4 is a detailed view of an embodiment of the disclosure.

FIG. 5 is a front view of an embodiment of the disclosure.

FIG. 6 is a cross-sectional view of an embodiment of the disclosure.

FIG. 7 is a cross-sectional view of an embodiment of the disclosure.

FIG. 8 is a detailed view of an embodiment of the disclosure.

FIG. 9 is a top view of an embodiment of the disclosure.

FIG. 10 is an in-use view of an embodiment of the disclosure.

FIG. 11 is a block view of an embodiment of the disclosure.

### DETAILED DESCRIPTION OF THE INVENTION

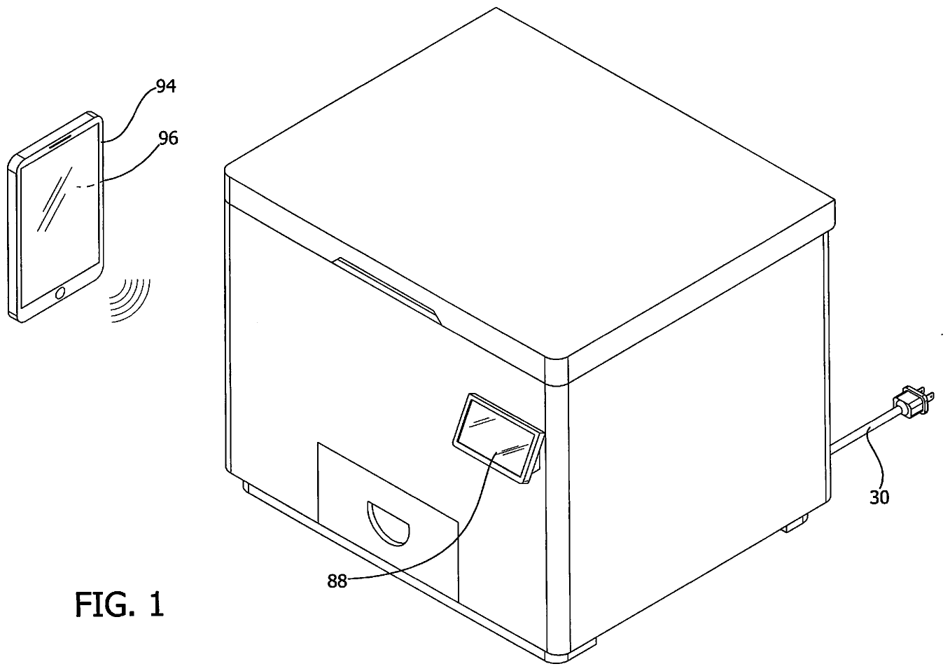

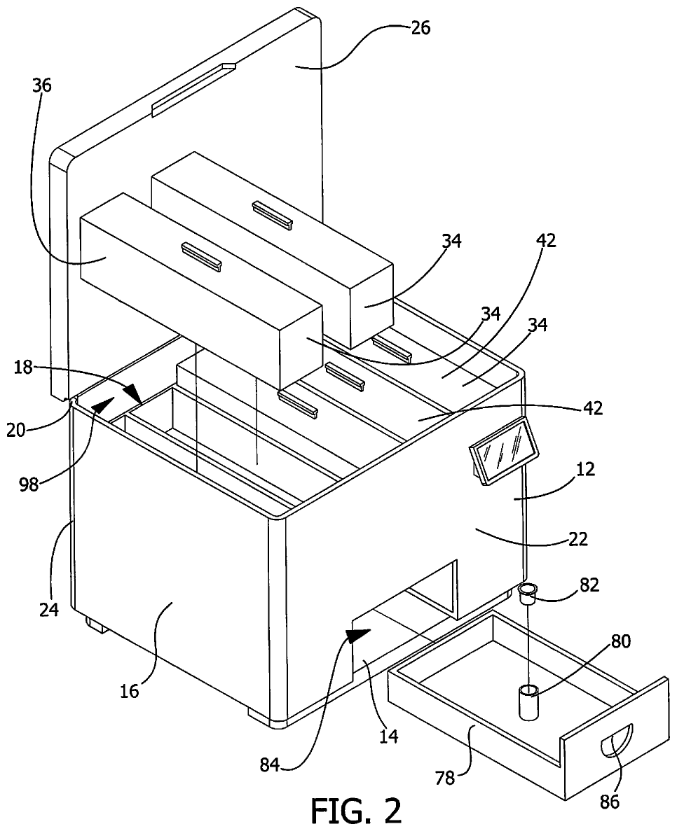

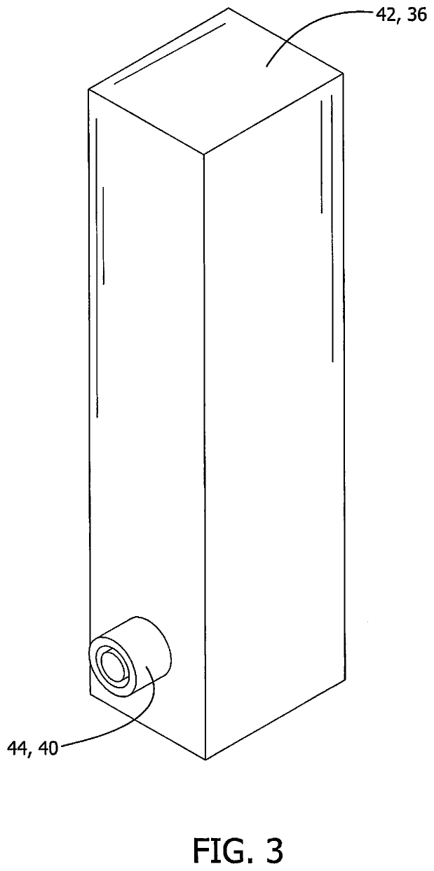

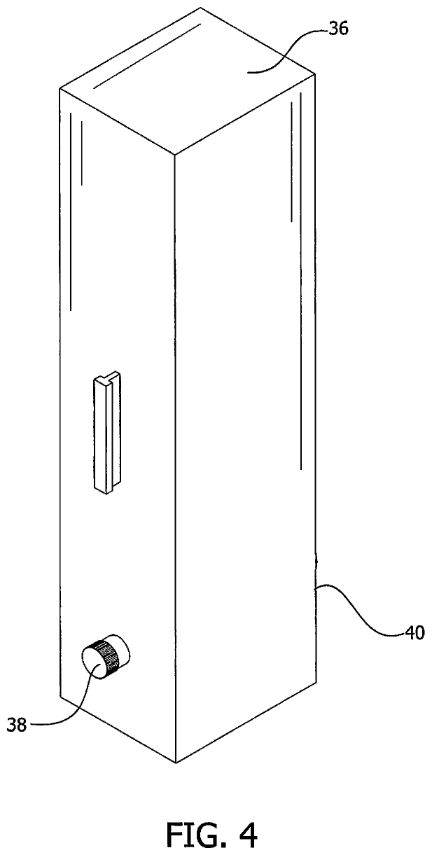

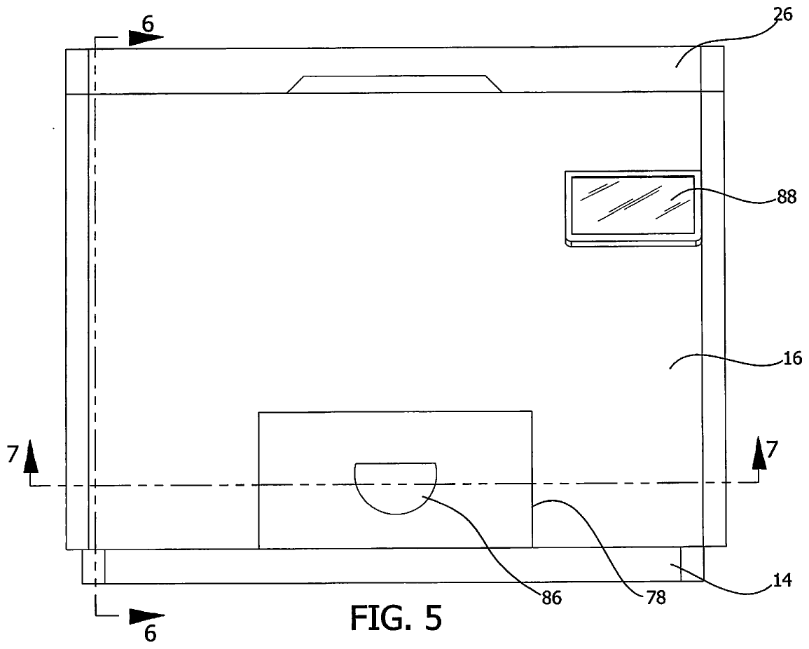

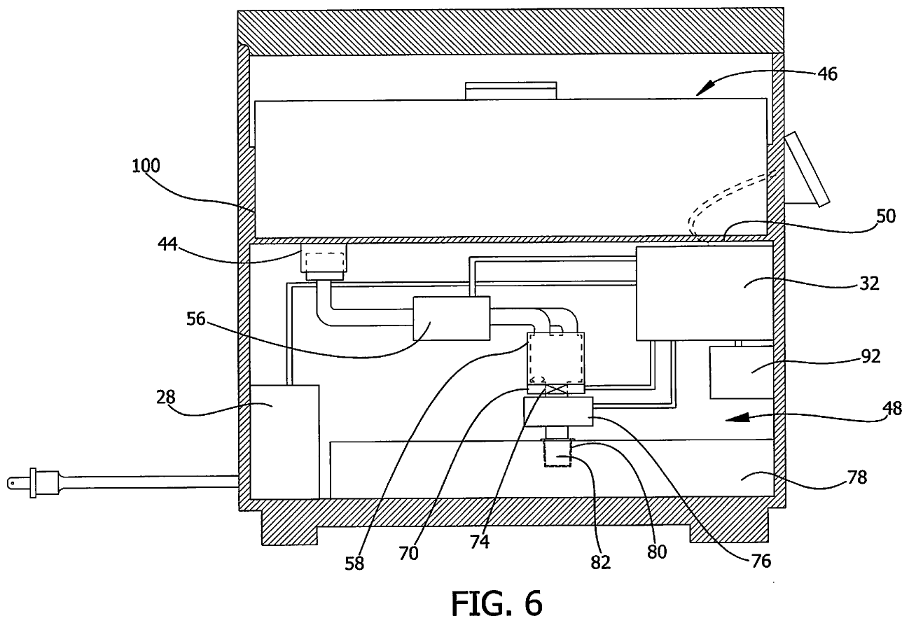

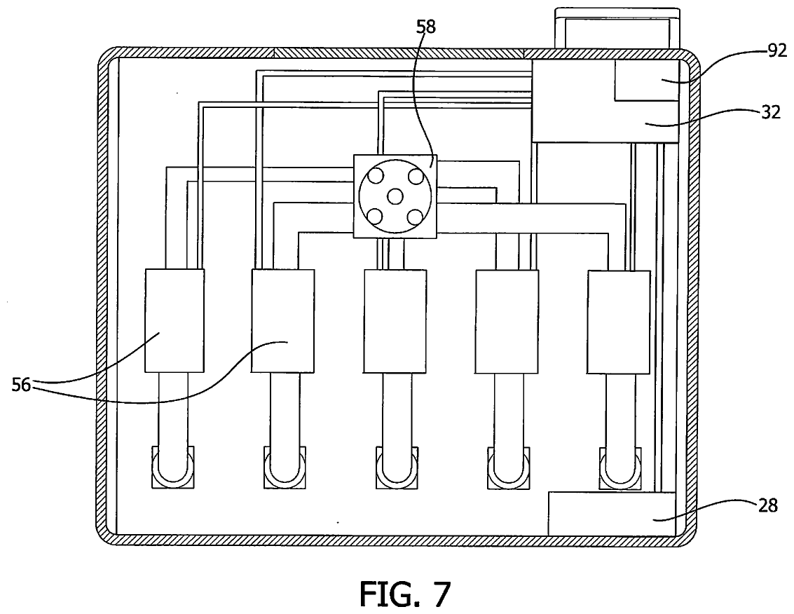

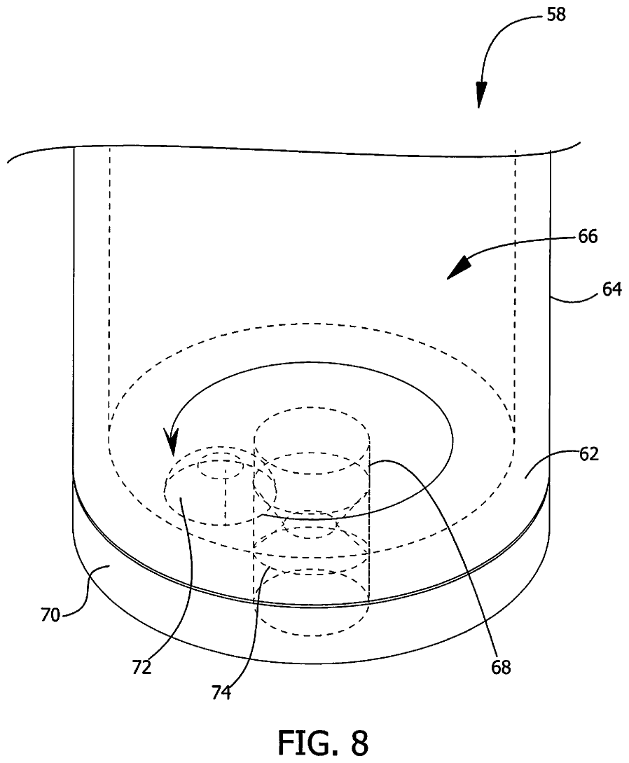

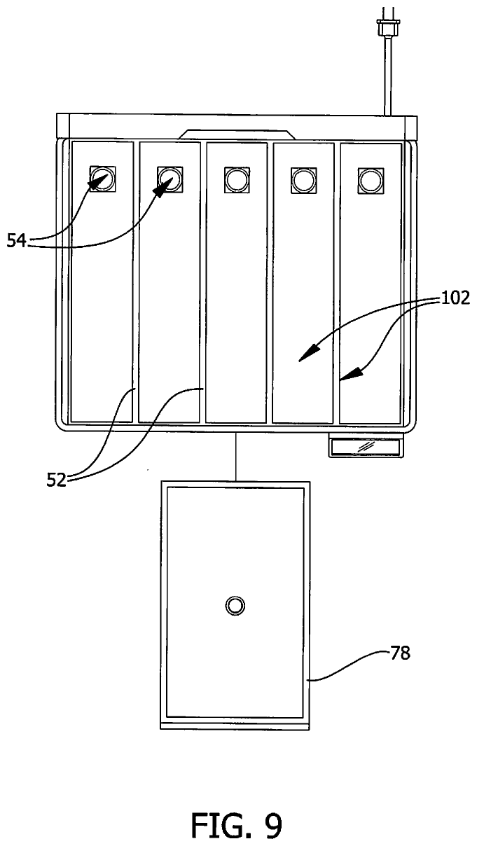

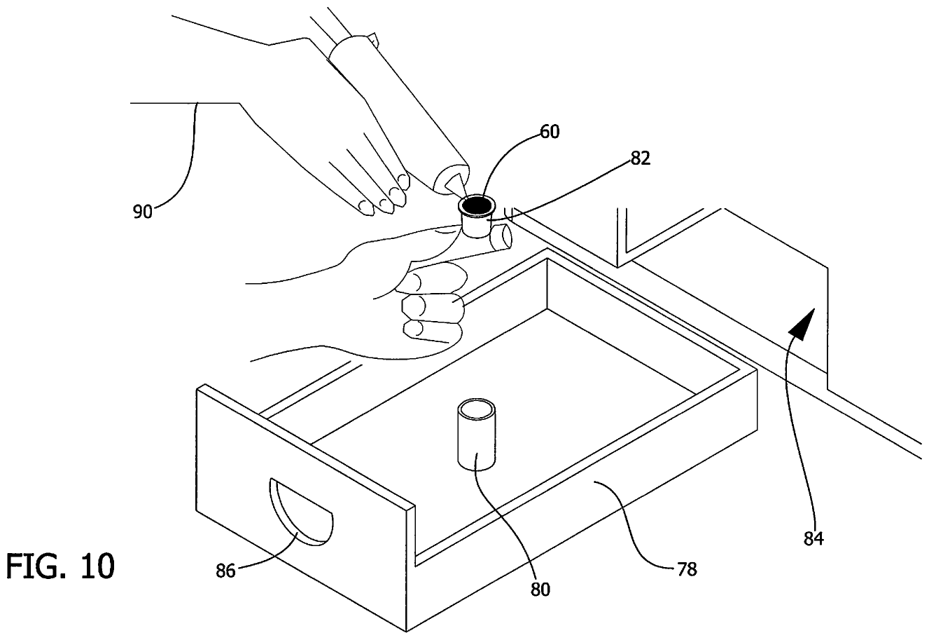

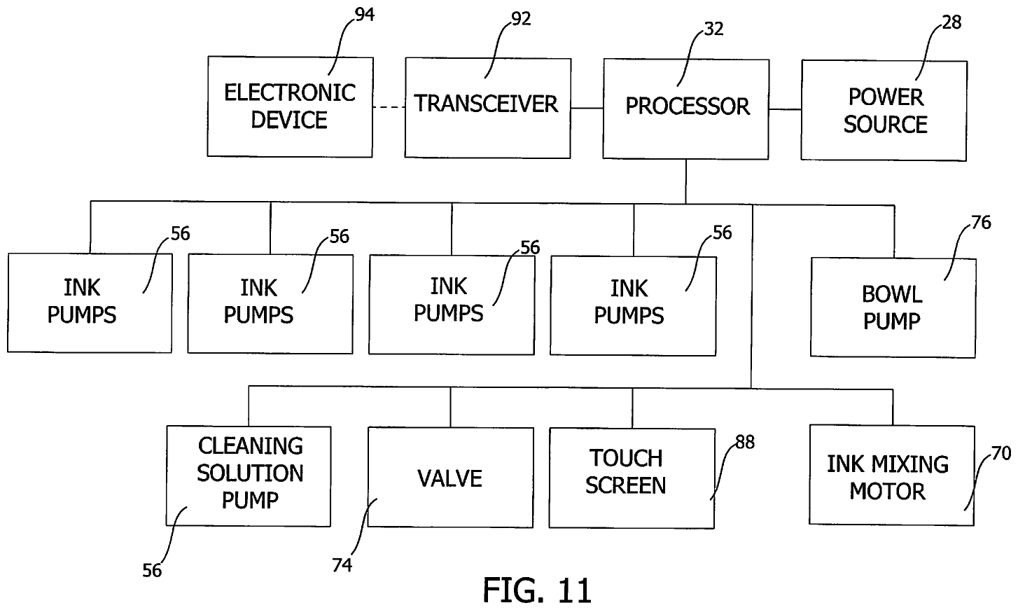

With reference now to the drawings, and in particular to FIGS. 1 through 11 thereof, a new ink blending machine embodying the principles and concepts of an embodiment of the disclosure and generally designated by the reference numeral 10 will be described.

As best illustrated in FIGS. 1 through 11, the tattoo ink mixing device 10 generally comprises a housing 12 having a bottom side 14 and a peripheral side 16 that is coupled to and extends upwardly from the bottom side 14 to define an interior space 18. The peripheral side 16 may have an upper edge 20 that is distal to the bottom side 14. The upper edge 20 may define an opening 98 into the interior space 18. The peripheral side 16 generally has a front wall 22 and a back wall 24.

A lid 26 may be pivotably coupled to the housing 12, for example being coupled to the back wall 24 of the peripheral side 16. The lid 26 may be pivotable downwardly toward the upper edge 20 to cover the opening 98 and facilitate access to the interior space 18. The lid 26 may be pivotable upwardly away from the upper edge 20 to expose the opening 98 and inhibit access to the interior space 18.

A power source 28 may be positioned in the interior space 18. The power source 28 may comprise a battery. A power cord 30 may be electrically coupled to the power source 28. The power cord 30 may be coupled to and extend outwardly from the housing 12, for example being positioned on the back wall 24 of the peripheral side 16.

A processor 32 is generally positioned in the interior space 18. The processor 32 is electrically coupled to the power source 28.

A plurality of cartridges 34 is positionable in the interior space 18. The plurality of cartridges 34 generally includes a set of ink cartridges 42. Each of the set of ink cartridges 42 holds a respective one of a plurality of colored inks. For example, the plurality of colored inks may include a cyan ink, a magenta ink, a yellow ink, and a black ink. Each of the plurality of colored inks are generally mixable with each other to produce various shades of a mixed ink 60. Each of the set of ink cartridges 42 may include an ink port 44 for releasing a quantity of the respective one of the plurality of colored inks. Each of the set of ink cartridges 42 may also include an ink input channel for refilling the respective one of the plurality of colored inks.

The plurality of cartridges 34 may further include a cleansing solution cartridge 36 that holds a cleaning fluid. The cleaning fluid is generally configured to remove any residue of the plurality of colored inks from the tattoo mixing device 10. For example, when the plurality of colored inks are water soluble, the cleaning fluid may comprise water. Certain embodiments of the plurality of colored inks may comprise dye, isopropyl alcohol, and distilled water, which is generally a water-soluble composition. When the plurality of colored inks comprise oils or other hydrophobic materials, an alternative cleaning fluid may be held within the cleansing solution cartridge 36.

The cleansing solution cartridge 36 may include a fluid inlet 38 for adding the cleaning fluid to the cleansing solution cartridge 36. The cleansing solution cartridge 36 may further include a fluid outlet 40 for releasing an amount of the cleaning fluid from the cleansing solution cartridge 36.

The interior space 18 may further comprise a first area 46 that is positioned proximate to the opening 98 and a second area 48 that is positioned beneath the first area 46 proximate to the bottom side 14. A panel 50 may be positioned in the interior space 18 to separate the first area 46 from the second area 48. For example, the panel 50 may be coupled to an interior surface 100 of the peripheral side 16.

The panel 50 may include a plurality of raised edges 52 extending upwardly into the first area 46 to define a plurality of slots 102. The plurality of slots 102 have a shape that is complementary to a shape of the plurality of cartridges 34 wherein a respective one of the plurality of cartridges 34 is positionable within each of the plurality of slots 102. A plurality of holes 54 may extend through the panel 50 into the plurality of slots 102. The plurality of holes 54 generally have a shape that is complementary to a shape of the ink port 44 and the fluid outlet 40 wherein the ink port 44 of the respective one of the set of ink cartridges 42 and the fluid outlet 40 of the cleansing solution cartridge 36 is positionable within an associated one of the plurality of holes 54.

A plurality of cartridge pumps 56 is positioned in the interior space 18, for example within second area 48 of the interior space 18. The plurality of cartridge pumps 56 is electrically coupled to the processor 32. Each of the plurality of cartridge pumps 56 is fluidly couplable to a respective one of the plurality of cartridges 34. Activation of each of the plurality of cartridge pumps 56 releases the quantity of the respective one of the plurality of colored inks and the amount of the cleansing fluid from the respective one of the plurality of cartridges 34.

A mixing bowl 58 is positioned in the interior space 18, for example being positioned within the second area 48 of the interior space 18. The mixing bowl 58 is fluidly coupled with each of the plurality of cartridge pumps 56 to receive the quantity of the respective one of the plurality of colored inks and the amount of the cleansing fluid. The quantity of the respective one of the plurality of colored inks mixes with an additional quantity of an additional one of the plurality of colored inks thereby forming a mixed ink 60.

The mixing bowl 58 may further comprise a base surface 62 and a perimeter surface 64 that is coupled to and extends upwardly from the base surface 62 to define an inner area 66. The inner area 66 receives the quantity of the respective one of the plurality of colored inks. A drain 68 may extend through the base surface 62 into the inner area 66 for releasing the quantity of the respective one of the plurality of colored inks. A valve 74 may be coupled to the drain 68. The valve 74 is generally movable between an open position and a closed position. The mixed ink 60 flows outwardly through the drain 68 when the valve 74 is in the open position. The mixed ink 60 is inhibited from flowing outwardly through the drain 68 when the valve 74 is in the closed position.

An electromagnetic motor 70 may be coupled to the base surface 62 of the mixing bowl 58. The electromagnetic motor 70 is electrically coupled to the processor 32. The processor 32 actuates the electromagnetic motor 70 to emit a magnetic field over the base surface 62. A magnet 72 may be positioned on the base surface 62 within the inner area 66. The magnet 72 may rotate around the base surface 62 when the electromagnetic motor 70 emits the magnetic field thereby mixing the quantity of the respective one of the plurality of colored inks with the additional quantity of the additional one of the plurality of colored inks to form the mixed ink 60.

A bowl pump 76 may be positioned in the interior space 18, for example being positioned within the second area 48 of the interior space 18. The bowl pump 76 is electrically coupled to the processor 32. The bowl pump 76 is fluidly coupled to the mixing bowl 58 to release the mixed ink 60 from the mixing bowl 58.

A tray 78 may be removably coupled to the housing 12. For example, the tray 78 may be slidable inwardly into the second area 48 of the interior space 18. The tray 78 is generally positioned beneath the bowl pump 76 when the tray 78 is positioned in the interior space 18.

A sleeve 80 may be coupled to and extend upwardly from the tray 78. The sleeve 80 may be aligned with the bowl pump 76 when the tray 78 is positioned in the interior space 18.

A cup 82 is generally removably positionable in the interior space 18. For example, the cup 82 may be removably positionable in the sleeve 80 on the tray 78. The cup 82 is fluidly coupled to the mixing bowl 85 via the bowl pump 76 when the cup 82 is positioned in the sleeve 80 and when the tray 78 is positioned in the interior space 18. The bowl pump 76 releases the mixed ink 60 into the cup 82. Typically, tattoo artists only use a very small amount of each shade of mixed ink 60 for a particular tattoo. For example, only a few milliliters of the mixed ink 60 may be required. Accordingly, the cup 82 may have a height between 0.2 inch and 0.6 inch and a diameter between 0.2 inch and 0.6 inch.

The tray 78 positions the cup 82 in fluidic communication with the bowl pump 76 and the mixing bowl 58 when the cup 82 is positioned on the tray 78 and when the tray 78 is positioned in the interior space 18. The sleeve 80 generally has a shape that is complementary to a shape of the cup 82 wherein the cup 82 is positionable within the sleeve 80 such that the sleeve 80 is configured to stabilize a position of the cup 80 on the tray 78. The sleeve 80 may also inhibit the cup 82 from moving, sliding, or tipping on the tray 78 as the tray slides inwardly and outwardly from the interior space 18.

A cutout 84 may extend into the housing 12. For example, the cutout 84 may be positioned on the front wall 22, proximate to a junction between the bottom side 14 and the peripheral side 16. The cutout 84 generally has a shape that is complementary to a shape of the tray 78 wherein the cutout 84 is configured to receive the tray 78 such that the tray 78 may be slid into the interior space 18 through the cutout 84.

A handle 86 may be coupled to the tray 78 to facilitate sliding the tray 78 inwardly and outwardly from the interior space 18.

The processor 32 is generally configured to selectively actuate each of the plurality of cartridge pumps 56, the electromagnetic motor 70, the valve 74, and the bowl pump 76.

A touch screen 88 may be coupled to the housing 12. The touch screen 88 is electrically coupled to the processor 32. For example, the touch screen 88 may be positioned on the peripheral side 16, such as on the front wall 22. The touch screen 88 may be angled upwardly with respect to the peripheral side 16. The touch screen 88 is configured to provide a troubleshooting interface for a user 90. For example, the trouble shooting interface displayed on the touch screen 88 may facilitate the user in checking levels of the plurality of colored inks or the cleaning fluid remaining in the plurality of cartridges 34. In another example, the trouble shooting interface may facilitate the user 90 in determining whether the plurality of cartridge pumps 56 and the bowl pump 76 are functioning as intended, or whether there is a clog in one of the lines fluidly coupling the plurality of cartridges 34 with the plurality of cartridge pumps 56 or the mixing bowl 58 with the bowl pump 76.

A transceiver 92 may be positioned in the interior space 18. The transceiver 92 is electrically coupled to the processor 32. An electronic device 94 may have positioned thereon a programming code 96 enabling the electronic device 94 for selecting a shade of the mixed ink 60. The electronic device 94 is in wireless communication with the transceiver 92 whereby the electronic device 94 communicates the shade of the mixed ink 60 to the transceiver 92. The processor 32 is configured to actuate the plurality of cartridge pumps 56 to release the quantity of the respective one of the plurality of colored inks and the additional quantity of the additional one of the plurality of colored inks into the mixing bowl 58 to form the shade of the mixed ink 60 when the electronic device 94 communicates the shade to the transceiver 92.

The processor 32 may actuate a particular one of the plurality of cartridge pumps 56 to dispense the amount of the cleaning fluid into the mixing bowl 58. The processor 32 may also actuate the bowl pump 76 to release the cleaning fluid outwardly through the bowl pump 76 to remove any residual amount of the mixed ink 60 from the mixing bowl 58 and the bowl pump 76 once the mixed ink 60 is released from the mixing bowl 58 through the bowl pump 76. This process may be called a wash cycle because the cleaning fluid washes out the mixing bowl 58 and the bowl pump 76 so that each of those components are clean before a new shade of the mixed ink 60 is produced. For example, the processor 32 may automatically actuate the particular one of the plurality of cartridge pumps 56 and the bowl pump 76 for the wash cycle. In some embodiments, the processor 32 may initiate the wash cycle once a certain amount of time has passed after the bowl pump 76 releases the mixed ink 60 from the mixing bowl 58. For example, the processor 32 may initiate the wash cycle a few minutes or seconds after the mixed ink 60 is released from the mixing bowl 58. Alternatively, the processor 32 may initiate the wash cycle manually. For example, the touch screen 88 mounted to the housing 12 may have a user interface that facilitates a user 90 in initiating the wash cycle. In another embodiment, the transceiver 92 may be positioned in the interior space 32 and electrically coupled to the processor 32. The transceiver 92 may be in wireless communication with the electronic device 94, and the programming code 96 on the electronic device 94 may enable the electronic device 94 for initiating the wash cycle.

In use, the user 90 can input the shade of the mixed ink 60, for example using the electronic device 94 or the touch screen 88. The processor 32 will actuate the plurality of cartridge pumps 56 to release the required amounts of each colored ink needed to match the shade of the mixed ink 60 chosen by the user 90. The colored inks will mix together into a uniform pigment within the mixing bowl 58 before being drained into the cup 82. The user 90 can remove the cup 82 from the interior space 18 and use the mixed ink 60 within the cup 82 for a tattoo.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An ink mixing system comprising:
a housing having an interior space;
a power source being positioned in the interior space;
a processor being positioned in the interior space, the processor being electrically coupled to the power source;
a plurality of cartridges being positionable in the interior space, the plurality of cartridges including a set of ink cartridges, each of the set of ink cartridges holding a respective one of a plurality of colored inks;
a plurality of cartridge pumps being positioned in the interior space, the plurality of cartridge pumps being electrically coupled to the processor, each of the plurality of cartridge pumps being fluidly couplable to a respective one of the plurality of cartridges wherein the processor activates at least one of the plurality of cartridge pumps to release a quantity of the respective one of the plurality of colored inks from the respective one of the plurality of cartridges;
a mixing bowl being positioned in the interior space, the mixing bowl being fluidly coupled with each of the plurality of cartridge pumps to receive the quantity of the respective one of the plurality of colored inks, wherein the quantity of the respective one of the plurality of colored inks mixes with an additional quantity of an additional one of the plurality of colored inks thereby forming a mixed ink; and
a cup being removably positionable in the interior space, the cup being in fluidic communication with the mixing bowl when the cup is positioned in the interior space whereby the cup receives the mixed ink.

2. The ink mixing system of claim 1, wherein the housing has a bottom side and a peripheral side being coupled to and extending upwardly from the bottom side to define the interior space, the peripheral side having an upper edge distal to the bottom side defining an opening into the interior space.

3. The ink mixing system of claim 2, further comprising a lid being pivotably coupled to the housing, the lid being pivotable downwardly toward the upper edge to cover the opening and facilitate access to the interior space, the lid being pivotable upwardly away from the upper edge to expose the opening and inhibit access to the interior space.

4. The ink mixing system of claim 2, the interior space further comprising:
a first area being positioned proximate to the opening;
a second area being positioned beneath the first area proximate to the bottom side; and
a panel coupled to an interior surface of the peripheral side to separate the first area from the second area.

5. The ink mixing system of claim 4, the panel further comprising a plurality of raised edges extending upwardly into the first area to define a plurality of slots, the plurality of slots having a shape being complementary to a shape of the plurality of cartridges wherein a respective one of the plurality of cartridges is positionable within each of the plurality of slots.

6. The ink mixing system of claim 1, further comprising:
a transceiver being positioned in the interior space, the transceiver being electrically coupled to the processor; and
an electronic device having positioned thereon programming code enabling the electronic device for selecting a shade of the mixed ink, the electronic device being in wireless communication with the transceiver whereby the electronic device communicates the shade of the mixed ink to the transceiver, the processor being configured to actuate the plurality of cartridge pumps to release the quantity of the respective one of the plurality of colored inks and the additional quantity of the additional one of the plurality of colored inks into the mixing bowl to form the shade of the mixed ink when the electronic device communicates the shade to the transceiver.

7. The ink mixing system of claim 1, the plurality of cartridges further comprising a cleansing solution cartridge holding a cleaning fluid, the cleansing solution cartridge including a fluid outlet for releasing an amount of the cleaning fluid from the cleansing solution cartridge.

8. The ink mixing system of claim 7, the cleansing solution cartridge further comprising a fluid inlet for adding the cleaning fluid to the cleansing solution cartridge.

9. The ink mixing system of claim 7, wherein the processor activates a respective one of the plurality of cartridge pumps to release the amount of the cleaning fluid from the cleansing solution cartridge, wherein the amount of the cleaning fluid is dispensed into the mixing bowl and outwardly into the cup to remove a residual amount of the mixed ink once the mixed ink is released from the mixing bowl.

10. The ink mixing system of claim 1, the plurality of colored inks including a cyan ink, a magenta ink, a yellow ink, and a black ink.

11. The ink mixing system of claim 1, each of the set of ink cartridges further comprising an ink port for releasing the quantity of the respective one of the plurality of colored inks.

12. The ink mixing system of claim 1, the mixing bowl further comprising:
a base surface;
a perimeter surface being coupled to and extending upwardly from the base surface to define an inner area, the inner area receiving the quantity of the respective one of the plurality of colored inks; and
a drain extending through the base surface into the inner area for releasing the quantity of the respective one of the plurality of colored inks.

13. The ink mixing system of claim 12, the mixing bowl further comprising a valve being coupled to the drain, the valve being movable between an open position and a closed position, wherein the mixed ink flows outwardly through the drain when the valve is in the open position, wherein the mixed ink is inhibited from flowing outwardly through the drain when the valve is in the closed position.

14. The ink mixing system of claim 12, the mixing bowl further comprising:
an electromagnetic motor being coupled to the base surface, the electromagnetic motor being electrically coupled to the processor, the processor actuating the electromagnetic motor to emit a magnetic field over the base surface; and
a magnet being positioned on the base surface, the magnet rotating around the base surface when the electromagnetic motor emits the magnetic field thereby mixing the quantity of the respective one of the plurality of colored inks with the additional quantity of the additional one of the plurality of colored inks to form the mixed ink.

15. The ink mixing system of claim 1, further comprising a bowl pump being positioned in the interior space, the bowl pump being electrically coupled to the processor, the bowl pump being fluidly coupled to the mixing bowl and the cup to release the mixed ink from the mixing bowl into the cup.

16. The ink mixing system of claim 1, further comprising a tray being removably coupled to the housing, the tray being slidable inwardly into the interior space, the tray being positioned beneath the mixing bowl when the tray is positioned in the interior space wherein the tray positions the cup in fluidic communication with the mixing bowl when the cup is positioned on the tray and when the tray is positioned in the interior space.

17. The ink mixing system of claim 16, further comprising a sleeve being coupled to and extending upwardly from the tray, the sleeve being aligned with the mixing bowl when the tray is positioned in the interior space, the sleeve having a shape being complementary to a shape of the cup wherein the cup is positionable within the sleeve whereby the sleeve is configured to stabilize a position of the cup on the tray.

18. The ink mixing system of claim 16, further comprising a cutout extending into the housing for receiving the tray.

19. The ink mixing system of claim 1, further comprising a touch screen being coupled to the housing, the touch screen being electrically coupled to the processor, the touch screen being configured to provide a troubleshooting interface for a user.

20. An ink mixing system comprising:
a housing having a bottom side and a peripheral side being coupled to and extending upwardly from the bottom side to define an interior space, the peripheral side having an upper edge distal to the bottom side defining an opening into the interior space, the peripheral side having a front wall and a back wall;
a lid being pivotably coupled to the back wall of the peripheral side, the lid being pivotable downwardly toward the upper edge to cover the opening and facilitate access to the interior space, the lid being pivotable upwardly away from the upper edge to expose the opening and inhibit access to the interior space;
a power source being positioned in the interior space, the power source comprising a battery;
a power cord being electrically coupled to the power source, the power cord being coupled to and extending outwardly from the back wall of the peripheral side;
a processor being positioned in the interior space, the processor being electrically coupled to the power source;
a plurality of cartridges being positionable in the interior space, the plurality of cartridges including:
a set of ink cartridges, each of the set of ink cartridges holding a respective one of a plurality of colored inks, the plurality of colored inks including a cyan ink, a magenta ink, a yellow ink, and a black ink, each of the set of ink cartridges including:
an ink port for releasing a quantity of the respective one of the plurality of colored inks;
a cleansing solution cartridge holding a cleaning fluid, the cleaning fluid comprising water, the cleansing solution cartridge including:
a fluid inlet for adding the cleaning fluid to the cleansing solution cartridge;
a fluid outlet for releasing an amount of the cleaning fluid from the cleansing solution cartridge;
the interior space further comprising:
a first area being positioned proximate to the opening;
a second area being positioned beneath the first area proximate to the bottom side;
a panel coupled to an interior surface of the peripheral side to separate the first area from the second area, the panel including:
a plurality of raised edges extending upwardly into the first area to define a plurality of slots, the plurality of slots having a shape being complementary to a shape of the plurality of cartridges wherein a respective one of the plurality of cartridges is positionable within each of the plurality of slots;
a plurality of holes extending through the panel into the plurality of slots, the plurality of holes having a shape being complementary to a shape of the ink port and the fluid outlet wherein the ink port of the respective one of the set of ink cartridges and the fluid outlet of the cleansing solution cartridge is positionable within an associated one of the plurality of holes;
a plurality of cartridge pumps being positioned in the second area of the interior space, the plurality of cartridge pumps being electrically coupled to the processor, each of the plurality of cartridge pumps being fluidly couplable to a respective one of the plurality of cartridges wherein activation of each of the plurality of cartridge pumps releases the quantity of the respective one of the plurality of colored inks and the amount of the cleansing fluid from the respective one of the plurality of cartridges;
a mixing bowl being positioned in the second area of the interior space, the mixing bowl being fluidly coupled with each of the plurality of cartridge pumps to receive the quantity of the respective one of the plurality of colored inks and the amount of the cleansing fluid, wherein the quantity of the respective one of the plurality of colored inks mixes with an additional quantity of an additional one of the plurality of colored inks thereby forming a mixed ink, the mixing bowl further comprising:
a base surface;
a perimeter surface being coupled to and extending upwardly from the base surface to define an inner area, the inner area receiving the quantity of the respective one of the plurality of colored inks;
a drain extending through the base surface into the inner area for releasing the quantity of the respective one of the plurality of colored inks;
a valve being coupled to the drain, the valve being movable between an open position and a closed position, wherein the mixed ink flows outwardly through the drain when the valve is in the open position, wherein the mixed ink is inhibited from flowing outwardly through the drain when the valve is in the closed position;
an electromagnetic motor being coupled to the base surface, the electromagnetic motor being electrically coupled to the processor, the processor actuating the electromagnetic motor to emit a magnetic field over the base surface;
a magnet being positioned on the base surface, the magnet rotating around the base surface when the electromagnetic motor emits the magnetic field thereby mixing the quantity of the respective one of the plurality of colored inks with the additional quantity of the additional one of the plurality of colored inks to form the mixed ink;
a bowl pump being positioned in the second area of the interior space, the bowl pump being electrically coupled to the processor, the bowl pump being fluidly coupled to the mixing bowl to release the mixed ink from the mixing bowl;
wherein the amount of the cleaning fluid is dispensed into the mixing bowl and outwardly through the bowl pump to remove a residual amount of the mixed ink once the mixed ink is released from the mixing bowl through the bowl pump;
a tray being removably coupled to the housing, the tray being slidable inwardly into the second area of the interior space, the tray being positioned beneath the bowl pump when the tray is positioned in the interior space;
a sleeve being coupled to and extending upwardly from the tray, the sleeve being aligned with the bowl pump when the tray is positioned in the interior space;
a cup being removably positionable in the sleeve, the cup being fluidly coupled to the bowl pump when the cup is positioned in the sleeve and when the tray is positioned in the interior space, the bowl pump releasing the mixed ink into the cup, the cup having a height between 0.2 inch and 0.6 inch and a diameter between 0.2 inch and 0.6 inch;
a cutout extending into the front wall of the peripheral side of the housing for receiving the tray, the cutout being positioned adjacent to the bottom side;
a handle being coupled to the tray to facilitate sliding the tray inwardly and outwardly from the interior space;
wherein the processor is configured to actuate the plurality of cartridge pumps, the electromagnetic motor, the valve, and the bowl pump;
a touch screen being coupled to the housing, the touch screen being electrically coupled to the processor, the touch screen being configured to provide a troubleshooting interface for a user, the touch screen being positioned on the front side of the peripheral side, the touch screen being angled upwardly with respect to the peripheral side;
a transceiver being positioned in the interior space, the transceiver being electrically coupled to the processor; and
an electronic device having positioned thereon programming code enabling the electronic device for selecting a shade of the mixed ink, the electronic device being in wireless communication with the transceiver whereby the electronic device communicates the shade of the mixed ink to the transceiver, the processor being configured to actuate the plurality of cartridge pumps to release the quantity of the respective one of the plurality of colored inks and the additional quantity of the additional one of the plurality of colored inks into the mixing bowl to form the shade of the mixed ink when the electronic device communicates the shade to the transceiver.
